(19) United States
(12) Patent Application Publication
(10) Pub. No.: US 2019/0216055 A1
(43) Pub. Date: Jul. 18, 2019

(54) PROCESS AND APPARATUS FOR DISBUDDING AND INHIBITING HORN GROWTH

(71) Applicant: GOLD RYTHMN PTY LTD, WARRAGUL (AU)

(72) Inventor: JOHN JAMES STEINFORT, WARRAGUL (AU)

(21) Appl. No.: 16/306,602

(22) PCT Filed: Jun. 1, 2017
(86) PCT No.: PCT/AU2017/000123

§ 371 Date: Dec. 3, 2018

(30) Foreign Application Priority Data
Jun. 1, 2016 (AU) 2016902120

(51) Int. Cl. A01K 17/00; A61B 18/02

(52) U.S. Cl. CPC A01K 17/00; A61B 2018/0047; A61B 18/0218

(57) ABSTRACT

A method of inhibiting the growth of a horn or a horn bud in a livestock animal including the steps of applying a cryogenic fluid around the base of the horn or on the horn bud using an apparatus; such that the cryogenic fluid contacts the base of the horn and epidermal skin layers that surrounds the horn or horn bud and induces a freeze in said layers to inhibit or prevent any further growth of the horn or horn bud.

### FIELD OF THE INVENTION

The present invention relates to a process and apparatus to inhibit the growth of horns and horn buds and to remove horn buds (disbudding) in livestock.

### BACKGROUND OF THE INVENTION

The presence of livestock horns function as a defence mechanism against predators and are utilised within herd or flock hierarchy. In modern day husbandry practices, horns or their precursors, horn buds, are generally removed from livestock at a young age. The term used is dehorning or disbudding. Homed cattle tend to injury each other, can be dangerous for livestock handlers and degrade their carcass value by the resulting bruising and hide damage from the horns.

Livestock, in particular domestic ruminants, can have permanent horns that tend to grow from horn buds at an early age and until maturity is reached. New horn tissue (keratin) is produced throughout the life of the animal, unlike deer, where their antlers tend to shed and then grow back again annually.

Modern day livestock management practices discourage the presence of homed cattle. Polled or hornless livestock, such as seen in sheep and cattle, carry a gene which is inherited and can result in polled progeny.

A common method of horn or bud (early horn stage) removal involves the use of a hot dehorning iron which heats and cauterizes the horn bud and surrounding epidermal or skin tissues. The heat burns and cauterizes both the horn bud and its surroundings and sometimes the covering skin, with the end result of skin covering and replacing the area which contained the horn bud. The animals are often tranquilized and given analgesics now to assist in their recovery, especially within the dairy industry.

The other most common method used in industry, especially in the northern pastoral areas in Australia which needs to be urgently addressed, is the process of cutting the horn or horn bud off the animal's head. A sharp knife excises the horn bud and surrounding skin tissues. This results in suffering of the animal, with blood loss, sometimes post infections and sometimes death. These animals are generally less than one year old and once dehorned in this manner, are much more susceptible to wild dog attacks and losses. The death rate in livestock from this dehorning process is approximately 2% in some areas of Australia. The setback to livestock is significant, however there is little data available. There is an urgent need for this process to be replaced by a humane method that reduces the process pain and post recovery pain and inflammation and has minimal animal impact without blood loss, infections and possible deaths.

The present invention seeks to overcome the adverse impact of the current methods and offers a humane and effective method of disbudding to inhibit further bud or horn growth, which will then align with current livestock management practices. The presence of a residual horn bud from this invention described may differentiate this practice from those current practices previously described.

### SUMMARY OF THE INVENTION

According to a first aspect of the invention, there is provided a method of inhibiting the growth of a horn or a horn bud in a livestock animal including the steps of:
applying a cryogenic fluid around the base of the horn or on the horn bud using an apparatus;
such that the cryogenic fluid contacts the base of the horn and epidermal skin layers that surrounds the horn or horn bud and induces a freeze in said layers to inhibit or prevent any further growth of the horn or horn bud.

Preferably the application of the cryogenic fluid to said horn base and skin layers results in a freezing and thawing of cells and tissues that make up said layers and thereby reducing or eliminating the vascular, cellular and neural integrity of the layers.

Preferably the cryogenic fluid contacting the layers is either in liquid form, gaseous form or a combination of a liquid and gaseous form.

Preferably the surrounding horn base and skin layers is subjected to a cryogenic fluid temperature of between minus 20 degrees Celsius and minus 80 degrees Celsius, preferably minus 50 degrees Celsius.

Preferably the method further includes applying the cryogenic fluid to the whole of the horn bud in a young livestock animal.

Preferably said horn base and surrounding epidermal skin layers after application of the cryogenic fluid, undergoes a healing process where there is a closed wound and where cellular necrosis and scar formation results.

Preferably the targeted skin layers surrounding the horn or horn bud is up to 25 mm wide.

Preferably said cryogenic fluid is liquid nitrogen or liquid air.

Preferably the method further includes determining the amount of cryogenic fluid to use based on the size of the horn or horn bud and the age, breed and size of the livestock animal.

According to a second aspect of the invention, there is provided apparatus for inhibiting the growth of a horn or a horn bud in a livestock animal including:

a vessel having a body through which a cryogenic fluid is applied around the base of the horn or on the horn bud;

such that the cryogenic fluid contacts the horn base and epidermal skin layers that surrounds the horn or horn bud and induces a freeze in said layers to inhibit or prevent any further growth of the horn or horn bud.

Preferably the apparatus further includes one or more apertures or passages through which the cryogenic fluid passes and is directed onto the horn base and the skin surrounding said horn or horn bud.

Preferably said body is open at an end which is placed over the horn or the horn bud.

Preferably the apparatus further includes seal means formed around said end to minimize or prevent the escape of the cryogenic fluid and to assist in directing the cryogenic fluid to said skin layers.

Preferably an opposite end to said end has connected thereto the supply of cryogenic fluid to the apparatus.

Preferably the apparatus further includes a vent hole to release any excess gas released from vaporization of the cryogenic fluid from within the body.

Preferably the apparatus further includes an interior member housing one or more outlets through which the cryogenic fluid flows, said one or more outlets angled so as to deliver the cryogenic fluid directly to the horn bud and the skin layers around and on said horn bud.

Preferably an opposite end to said end is also open to enable the horn of the animal to protrude.

Preferably the apparatus further includes a ring to fit within the confines of the body, said ring having one or more apertures through which flows the cryogenic material.

Preferably said one or more apertures are directed downwardly at between 10 degrees and 65 degrees, preferably at about 45 degrees, with respect to the circular axis of the ring in order to target the skin layers with the cryogenic fluid.

Preferably the apparatus further includes a manifold, one side of which is connected to a supply of the cryogenic fluid and an opposite side connected to one or more members that are also connected to said ring to deliver the cryogenic fluid to the ring.

Preferably the ring is spaced apart from an interior of the body.

Preferably the apparatus further includes handle means to assist a user in applying the apparatus to the animal with sufficient force.

Preferably a further apparatus having a body smaller in diameter to said apparatus and also having a seal means is able to be fitted within said apparatus and below the ring in order to accommodate for a different sized horn.

Preferably said cryogenic fluid is applied for between 1 second and 10 seconds.

The invention in one embodiment describes a method of applying a cryogenic fluid to the base of the horn or horn bud via apparatus that directs the cryogenic fluid onto the targeted area. The freezing and thawing action will produce a cellular lysis and disruption which will directly inhibit further horn or horn bud development and growth.

In a preferred embodiment, the horn is produced by a layer of skin cells or epidermis at the base of the horn or horn bud. This layer of skin surrounds the horn bud base and produces the substance of the outer horn, known as the epiceras. The horns in domestic ruminant animals are permanent and grow continuously throughout their life. The inner horn component is an extension of the caudal frontal sinus and is initially formed of cartilage and then, as the animal matures, forms bone which is the base structure of the horn. The blood supply to the horn epiceras (horn bud state) or bone is from the continuation of the frontal sinus and from the inner sinus layers.

In a preferred embodiment, the cryogenic fluid is applied to the whole circumference area at the base of the horn or horn bud (lower horn periphery and horn skin base) and is applied for a period of time to affect a deep tissue freeze. The depth of freeze is preferred to be minus 50 degrees Celsius and can be from minus 20 to minus 80 degrees C. The amount of time to apply the cryogenic agent or as a preferred embodiment of the invention, liquid nitrogen or liquid air, varies and the average time per horn base is from 1 to 10 seconds, with average application time of 5 seconds per horn or horn bud base.

As an example, the preferred apparatus for inhibiting horn growth has a circular, conical or elongated conical shape, with a tube connecting the liquid nitrogen or liquid air to the apparatus. The edge of the base of the cone-shaped or circular-shaped receptacle with a seal interface is placed over the horn bud, ensuring that the size of the base of the apparatus fully covers the horn or horn bud base. The horn or preferably at a younger age, the horn bud base tissue, is saturated with liquid nitrogen or liquid air to produce a deep cryogenic freeze of approximately minus 50 Degrees Celsius, with resulting cellular lysis, with inhibition of horn or horn bud growth.

The avascular necrosis and cellular lysis results from the thawing process of the cells of the targeted epidermal layer, where vascular, neural and cellular integrity becomes dysfunctional, resulting in a closed wound which heals slowly at the cryogenic liquid application site. A combination of scar and surrounding skin tissue replace the epidermal horn ring. The production of short papillae, which constitute the outside horn structure, subsequently ceases. The cryogenic freeze may also affect the underlying targeted cornual base process which forms the structural horn base. The cornual process in younger livestock is cartilaginous in nature and ossifies to forms bone matrix as the animal matures and as the horn grows. The cryogenic freeze of about minus 50 Degrees C. around the circumference of the cornual base, results in a cryogenic induced necrosis with a disruption of cellular organisation resulting in vascular and neural shutdown. This process results in an effective dislocation or disassociation of the residual horn bud or horn forming the horn base. The remaining horn or horn bud remnants are movable to various degrees, within the connecting skin whilst often losing their skeletal base connection. This phenomenon is sometimes seen in cattle and the moveable horns or residual horns are known as scurrs.

The end results of this process may be differentiated from traditional means. In the process described herein there may be remaining residual horn or horn bud remnants, however they appear stunted as no further horn or bud growth has occurred. Whereas with conventional means, generally these processes remove the majority or total horn bud or horn growth, and where the replacement tissue is from surrounding skin, producing an area being covered by skin and hair. Dehorning conducted by dehorners or cutting instruments usually excise the whole horn or can sometimes remove most of the horn, leaving a section of horn base showing as cut horn, which remains connected to the cornual or horn base of the skull, and is rigid and fixed as such.

In one embodiment of the present invention, the animal is restrained generally in an upright position, such as a traditional head bale and crush. Multi modal analgesics can be administered prior to the process to give pain relief at and immediately post application. However in the present invention once the thawing process has been completed, there will be a loss of nerve innervation to the targeted site and the residual pain is then expected to be very low. Therefore, the process has minimal or no impact on the livestock, due to the de-innervation of the nerves within the targeted areas, the resultant slow healing process where the targeted skin and underlying structures no longer have effective blood supplies and the body then produces a slow tissue rejection process, that is characterised by a closed healing area and with targeted skin contraction and eventually skin slough as a scab.

The process preferably involves liquid nitrogen or liquid air being drawn into the line of the application means under pressure, whereby in a preferred embodiment an expandable and circular apparatus is placed over the one horn bud base, on and below so as to include the junction of the skin and horn bud base.

In an embodiment a solenoid valve or trigger mechanism, such as a gate valve, is activated to release the liquid nitrogen or liquid air into the application means and resultant fluid nitrogen/air which contains both liquid and vapour, but preferably a higher percentage of the liquid component, is released and exposed directly to the targeted skin area at the circular or oval horn base. The cryogenic fluid has direct contact with the epidermal skin that forms the horn or horn base thereby freezing the targeted skin area to preferably minus 50 degrees Celsius, as measured on the surface temperature of the skin of the animal. The cryogenic effect penetrates the deeper layer of the horn bud base and affects a deep freeze of approximately similar temperature.

In another embodiment of the invention, the whole of the horn bud as well as the surrounding skin junction may also be exposed to the liquid cryogen. If this occurs the horn bud will also freeze, resulting in the avascular necrosis with cellular lysis of the epiceras area of the horn or horn bud. If the majority of the horn bud undergoes an avascular necrosis, the horn bud may contract, shrink and may eventually slough after several weeks and be replaced by scar and/or epidermal tissue, with an eventual hair or wool cover.

In contrast, if only the periphery of the harder horn or horn bud is targeted with the present invention process, and this exposes these tissues to the freezing process induced by the application of the cryogenic liquid, the horn bud may not grow further and may remain as a small and slightly moveable horn bud stump and effectively like a residual horn or horn bud.

According to a further embodiment of the invention there is provided apparatus for inhibiting horn bud growth, including applicator means for applying the liquid nitrogen or liquid air onto the epidermal layer at the immature horn or horn bud base, where the applicator means does not allow spillage of the cryogenic fluid on surrounding tissues and structures.

Positive pressure is preferably applied by the operator on the apparatus to the horn bud base, when applying to the targeted area, and an effective seal is formed as a result. Preferably the cryogenic fluid contacts directly the horn bud base and delivers a cryogenic freeze of approximately minus 50 degrees Celsius.

Preferably the horn bud growth is inhibited and a residual horn bud or stump remains in place;

According to yet a further embodiment of the invention, there is provided apparatus for inhibiting horn bud growth, including where the process apparatus is placed over the entire horn bud (as would be the case in very young animals) and the cryogenic fluid is applied and makes contact to the entire area enclosed by the process application means. The horn bud and surrounding epidermis and sometimes covering skin of the horn bud, undergo the process application procedure where a cryogenic fluid is applied for 1 to 10 seconds. The cryogenic fluid makes contact with the horn bud and surrounding horn bud skin perimeter, whereby an avascular necrosis process or cellular lysis takes place, with resultant scar tissue over a 2 to 12 week period, resulting in the effective loss of the horn bud and replacement by skin, hair or wool and some scar tissue, without further horn bud growth due to the loss and necrosis of the horn bud growing epidermal tissues.

This results in avascular necrosis and effective absorption of the affected tissues and structures, and can remain as a closed wound during the healing process to be eventually replaced by skin and or scar tissue with surrounding hair or wool.

According to a further embodiment of the invention, there is provided a method for inhibiting horn growth in ruminants, the method including where:

the application apparatus to the skin is generally conical or circular or resembles the horn or horn bud base size,

a system of lines and pipes supply the cryogenic agent:

the application apparatus is held firmly in place and cryogenic fluid is applied for a predetermined time period;

the application apparatus varies in sizes for different livestock age and horn bud or horn sizes, of which a line is connected to each with an independent trigger or solenoid valve release system.

Preferably, when the process is applied to the base of a horn, the epidermal skin layer and some of the horn base is fully enclosed within the apparatus. A cryogenic fluid is then applied via the application means and is applied from 1 to 10 seconds. The cryogenic fluid makes contact and aims to have thorough dispersion within the application equipment, affecting a cryogenic freeze of about minus 50 Degrees Celsius, but generally with a range from minus 20 to minus 80 degrees Celsius.

The horn base structures freeze temperature is approximately minus 50 degrees Celsius with a range of minus 20 to minus 80 degrees Celsius.

Thus, the resultant cryogenic impact will initially freeze the targeted areas. The subsequent tissue fluid crystal thawing effect will result in cellular lysis and dysfunction of the neural and vascular systems affecting the targeted tissues. The area of cornual epidermal base targeted will depend upon the animal's age and outer dimensional size of the application apparatus. The circumference width of targeted tissue can vary from 2 mm to 10 mm. The average is approximately 5 mm skin width.

The surrounding epidermal cells, which produce the horn substrate, undergo a necrosis process through the freezing and thawing action.

The process may involve the one single application of the cryogenic fluid, being nitrogen or air. Alternatively it may involve two or three subsequent applications of the cryogenic fluid.

For the purpose of this specification, horn bud and horn describes the different age and growth stage where a horn bud is either the early residual or the early growth stage of a horn, which is still relatively new and has not gained much length.

The cryogenic fluid can produce a cryogenic freezing effect on the horn bud base epidermal cells and on the horn or horn bud tissue.

The apparatus supplying the cryogenic fluid is delivered via the system, which can house between 1 to 10 litres at the closest delivery point to the horn or bud base. This will produce an almost immediate flow of the liquid component to the site of application.

The cryogenic fluid is preferably delivered in mostly liquid form.

The delivery of the cryogenic fluid to the disbudding apparatus is preferably controlled by valves, which can be solenoids or other valve systems. The cryogenic fluid is preferably applied for between 1 to 10 seconds, more preferably 3 to 5 seconds.

The end resultant surface temperature of the freeze is approximately minus 50 degrees Celsius, and can range from minus 20 to minus 80 degrees Celsius: This is a desired effect as it will minimise pain in immediate and post recovery stages and the process generally does not involve any open wounds, so there is very minimal risk of infections, blood loss and risks of wild dog attacks.

According to a further embodiment of this invention, there is provided a method of applying the cryogenic fluid in such a manner that results in a cryogenic freeze and eventual necrosis of the tissues targeted. The method preferably includes applying one or both disbudding apparatus to the whole circumference base of the horn bud or horn;

applying the cryogenic fluid to the epidermal horn base and can include the horn structure.

Applying the cryogenic fluid to the horn or horn bud base or section thereof to produce an effective deep freeze and subsequent slow thaw may comprise achieving a temperature of approximately minus 50 (fifty) degrees Celsius on the targeted area. The processed area will feel solid after the freezing agent (cryogenic fluid) has been applied. Optimally, the temperature of the targeted tissues is between minus 20 degrees Celsius and minus 80 degrees Celsius, with an optimal average of minus 50 degrees Celsius.

The cryogenic fluid may be applied topically. The cryogenic fluid may comprise liquid nitrogen or liquid air. A mix of liquid and vapour can be applied however it is preferable the liquid component be the dominant component so as to effect a faster freeze.

The method is mostly bloodless and non-invasive when compared with surgical excision or dehorning cutting instruments which leave an open wound, hot dehorners with heat cauterization and with resultant painful skin burning.

The healing stages of both methods minimise the exposure of the animal to predators (wild dogs) as the wound is generally closed, with minimal infections and minimal evidence of blood. The healing stage involves minimal pain due to the de-innervation of the nerves at the local targeted site. The result may improve the recovery process and therefore have far less impact on the animal. Weight gains and mothering are all important features and favoured results for any procedure with livestock.

The methods may involve the application of the cryogenic fluid at a rate of between 50 and 300 gms of liquid component of the liquid nitrogen or air for between 1 to 10 seconds. Preferably optimum practice is to apply approximately 150 to 250 gms of the cryogenic fluid, for example liquid nitrogen in approximately 3 to 5 seconds. A similar procedure and application rate can be applied to the adjacent horn bud and its skin base. It is possible to apply two separate apparatuses, one to each horn bud simultaneously. The amount of cryogenic fluid will depend upon the age, breed and amount of horn bud or growth. It is preferable to apply the cryogenic fluid quickly and preferably between 1 to 10 seconds, again depending upon the animal type and age differentials.

The cryogenic fluid may be part liquid and part vapour form as it contacts the horn base, but preferably mostly a liquid component. The cryogenic fluid may be applied continuously on to the skin.

The cryogenic fluid is preferably applied to the horn bud or horn base layers via a delivery system via specialised conical or cylindrical shaped delivery apparatus. The storage and delivery of the cryogenic fluid can be via a portable storage vessel set at a relatively low pressure of between 2 psi and 40 psi or preferably 5 to 10 psi and holding from 10 kg to 450 kg. There may be a smaller holding vessel at the point of delivery of the liquid nitrogen/air via the solenoid or valve system. Accidental skin spillage must be prevented and a special holding and restraint system may be used and preference for the animal to have its head in the vertical plane, whether upright or on their backs with head being in the vertical plane.

The animals can be restrained in either an upright, standing and restrained position or on their backs or on their sides.

The cryogenic fluid may be sprayed or applied through spray means, such as single or multiple spray ports and can be contained within the end dispense apparatus means.

These methods described can be used to process animals as part of or replacement of current husbandry procedures and the system processing times may be similar to that that is currently achieved under current systems.

### BRIEF DESCRIPTION OF THE DRAWINGS

The preferred embodiments of the invention will hereinafter be described, by way of example only, with reference to the drawings in which:

FIG. 1A is an overhead view of a young calf s head with two horn buds:

FIG. 1B is a close up perspective view of two horn buds as would be viewed on a Bos Indicus or Brahman type calf;

FIG. 1C is a side view of a horn bud;

FIG. 2A is a cross-sectional view of a frontal top part of a calf's skull showing the horn and associated structures:

FIG. 2B is a similar cross sectional view to FIG. 2A of part of a calf skull and horn and shows the targeted skin after the cryogenic application;

FIGS. 3A and 3B are side view of apparatus for applying cryogenic fluid as would be applied to horn buds and small horns:

FIG. 3C is bottom end view of FIG. 3B;

FIG. 3D is a perspective and underneath view of an alternative application means or apparatus particularly suited to applying cryogenic fluid to a horn bud;

FIG. 3E is a perspective view of an alternative application means or apparatus particularly suited to applying cryogenic fluid to the base around an existing horn and shows a further fitting or vessel to cater for smaller homed animals;

FIGS. 4A and 4B are both perspective views of FIGS. 3A and 3B of the apparatus as applied around the circumference of a horn or horn bud:

FIGS. 5A and 5B are block diagrams of embodiments of a system used to apply a cryogenic fluid so as to inhibit horn growth;

FIGS. 6A and 6B are block diagrams of further embodiments of a system for applying cryogenic fluid to horn buds or horns on young livestock to inhibit the growth of the horn buds or horns;

FIG. 7A is a perspective view of a further embodiment of a mobile delivery system for enabling application of cryogenic fluid to horns or horn buds of livestock animals:

FIG. 7B is a perspective view of a smaller portable unit compared to FIG. 7A and

FIG. 7C is a perspective view of a system for delivering cryogenic fluid to multiple outlets or ports where each outlet or port is used to apply cryogenic fluid to horns or horn buds of respective livestock animals.

### DETAILED DESCRIPTION OF THE PREFERRED EMBODIMENTS

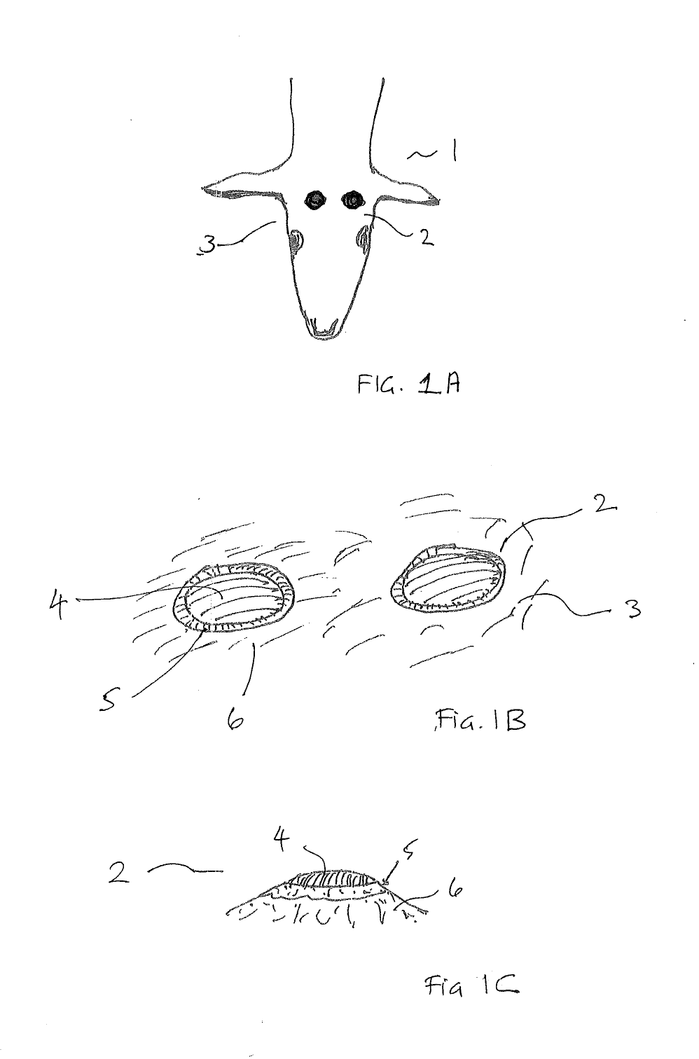

Referring to FIGS. 1A, 1B and 1C, there are shown differing embodiments and perspectives of horn buds, as would be seen in young ruminant livestock. FIG. 1A shows a calf's head and neck 1 with two round and approximately similar sized horn buds 2, positioned on a top frontal head area 3 of the calf. The roundness of the horn buds represented is typical of young horn buds seen in dairy and Bos Taurus animal types. FIG. 1B represents the frontal cranial area 3 of a Bos Indicus calf, where the horn buds 2 are elliptical and the harder keratin section of the horn bud is slightly raised. The horn bud has surrounding skin 4 at the bud base. This is a layer of surrounding skin that produces and supports the growth and development of the horn bud and subsequent horn. Surrounding skin and hair 6 is indicated.

FIG. 1C is a side view of a horn bud from FIG. 1B. The dome effect of the horn bud 4 is illustrated and surrounding base skin layer 5, which is connected to the general skin 6 of the head.

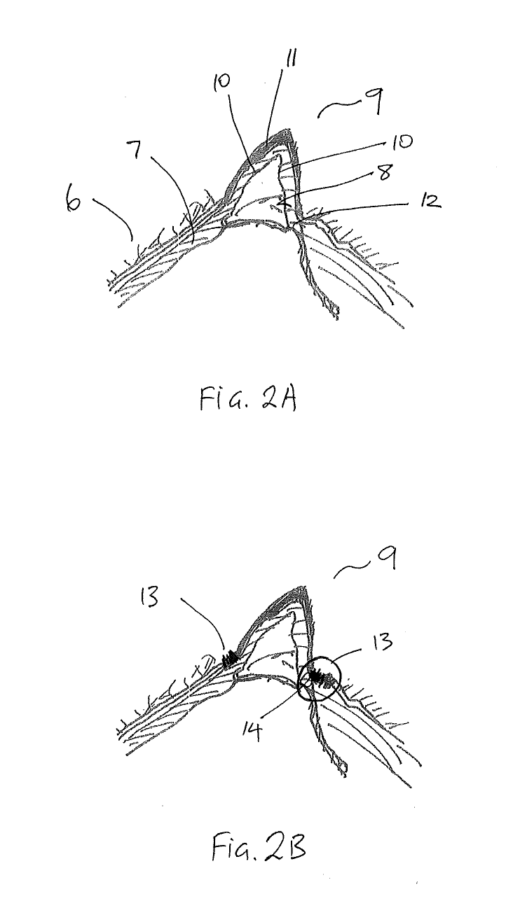

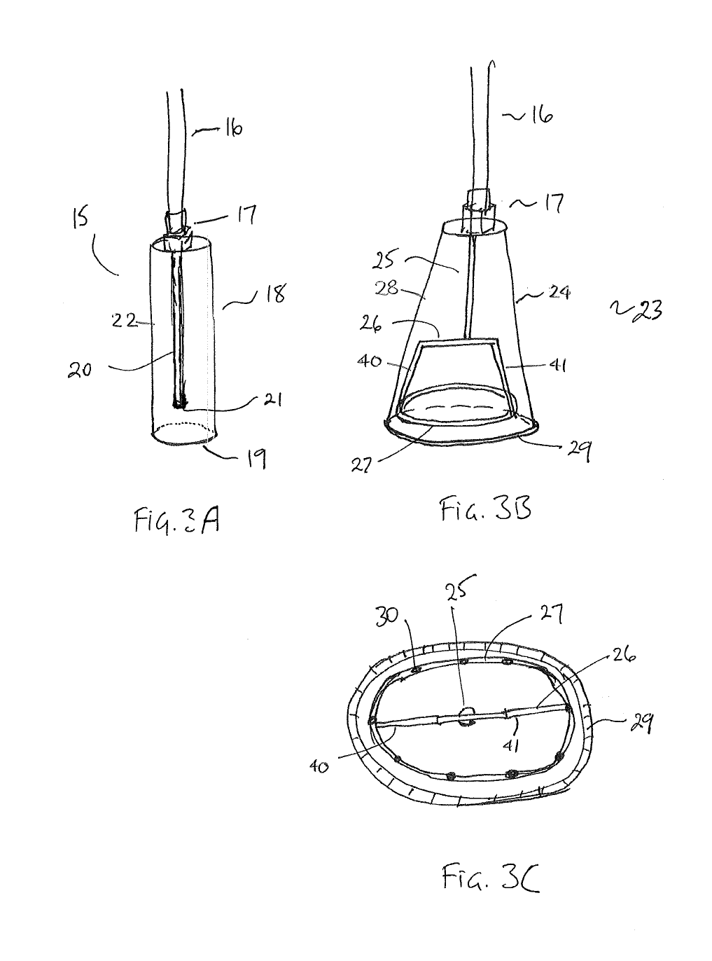

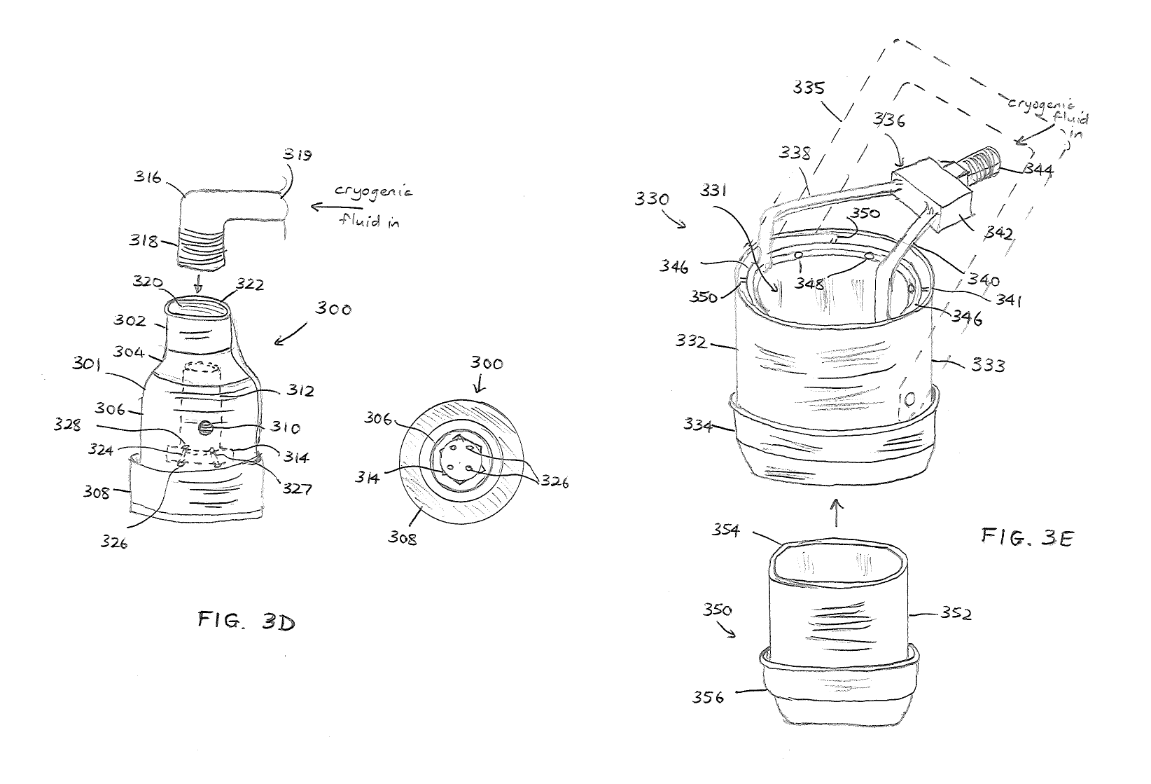

Referring to FIGS. 2A and 2B, there are shown two cross-sectional views of the top section of the cranium of a ruminant that is several months of age. There is seen some horn development. The surrounding skin and hair 6 is associated with the frontal bone 7, which extends to the cornual process 8, forming the cartilaginous and subsequently bony structure 10 of the horn 9. The cornual process 8 has an open cavity within known as the caudal frontal sinus, and is interconnected with the frontal sinuses of the skull. The horn epiceras produced by the epidermis at the base is initially soft and mostly contains keratin. In many ruminant species there is an ossification centre at the cornual junction 12, from which the bone component of the horn grows.

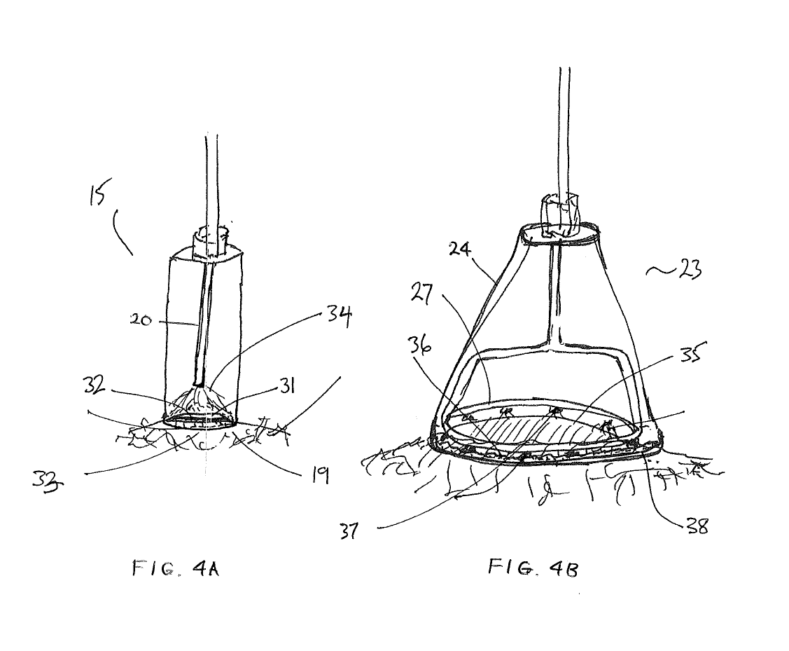

FIG. 2B is a similar cross sectional image of the horn and skull structures and illustrates the targeted anatomical area and the immediate structures affected after the cryogenic fluid or liquid application process. The apparatus (shown in FIGS. 3A and 3B) freezes the horn base 13 (encircled). The immediate epidermal horn base undergoes the cryogenic process, with resultant cellular lysis followed by significant reduction to produce further horn growth. The deeper structures including the growth plate of the ossification centre 14 are also cryogenically targeted, being part of the horn bud base, and affected resulting in inhibition of further bony horn growth. The depth and precise location of cryogenic action determines whether the residual horn, which has lost its ability to grow, will appear to be disassociated or attached to the skull.

In relation to FIGS. 3A and 3B, there is shown a block diagram of apparatuses or application means 15 and 23 that are used to apply cryogenic fluid, like liquid nitrogen or liquid air, to the horn bud and/or circumference or base of the horn bud or small horn. FIG. 3A shows apparatus 15 includes a hose 16 which carries liquid nitrogen or liquid air from its storage source to the vessel of application apparatus 15. The hose is connected via series of connectors 17 to a first top end of a cylindrical pipe-like vessel 18 which has an opposing open bottom end 19. The cryogenic liquid is transferred along tube 20, which is set at differing lengths inside the vessel 18 and released through outlet 21 of tube 20. The open end or aperture 19 is set for each apparatus unit and is designed for young calves, young goats or lambs at a certain young age As an example, dairy calves of 3 to 6 weeks or dairy goats of a few days old. The aperture 19 can have a range of diameters to suit the particular age group and subsequent horn bud size. The aperture 19 size range can be from 1.0 cm diameter to 10 cm diameter. The apparatus can have a circular or oval like circumference.

Generally, with very young animals, the horn bud is very small and may be covered by skin. The apparatuses 15, 23 are designed to go over the skin covering the horn bud or the protruded horn bud and to include the surrounding horn bud base skin so as to ensure the outer horn bud circumference, the horn bud and or skin covering the horn bud is targeted with the exposure of liquid nitrogen or liquid air directly upon these described areas. In this embodiment of the apparatus 15 in FIG. 3A, the whole horn bud and surrounding horn bud base will have contact with the cryogenic fluid.

FIG. 3B shows an embodiment of the apparatus or application means 23 to apply cryogenic fluid or liquid to the surrounding horn bud base and is designed for larger horn buds which may be maturing into small horns, such as is seen in older calves. In particular as seen in older Bos Indicus calves, where their horn buds tend to have a wide base circumference and are generally oval shape, with only minimal height. In this embodiment of the apparatus 23, there is an inlet hose 16, which transfers the cryogenic fluid through a series of connectors 17 to a tube 25 which is internal to outer walled section of frusto-conical vessel 28 of the apparatus 23. The outer lateral wall 24 of vessel 28 can also be conical or cylindrical in shape. The internal tube 25 can bifurcate into two parts 26, either at right angles or as a Y section. The bifurcated tubes 40, 41 are connected to a circular or oval shaped tube or ring 27, where outlets or small apertures 30 are positioned on the underneath aspect of tube 27. The outer walls 22, 24 with a flexible seal attached at 19 and 29 (not shown) in the embodiments of the apparatuses 15 and 23 serve to shield the cryogenic liquid within the respective vessel, and minimise any spillage or leakage under outer rim 29 (or end 19) and also to provide a means to hold or manoeuvre the apparatus for placement and location above the horn buds. Handles 335 can be provided for on the outer walls 22, 24. The apparatuses 15, 23 can be made from steel, plastic and their combinations. The longitudinal diameter can average approximately 10 cms and the adjacent diameter average being about 8 cms.

FIG. 3C is a block diagram and underneath view of FIG. 3B. The outer rim 29 contains the cryogenic liquid, which is transported via internal tube 25 through the bifurcation tubes, 40 and 41 into the perimeter tube 27. The release apertures 30 are positioned often sequentially around the perimeter tube 27.

Referring to FIG. 3D there is shown a front perspective view and an end view of apparatus that can be applied to a livestock animal to disbud or inhibit the horn growth on the animal. The apparatus or application means 300 has a vessel 301 which is bottle-shaped having a head 302, neck 304 and body 306. This embodiment is particularly for calves. On the inside of the head 302 and starting at the top end 320 of the head there is a threaded portion 322 which mates with a corresponding threaded portion 318 on the bottom of an elbow piece 316. The other end of the elbow piece 319 accepts cryogenic fluid and this is then delivered to the apparatus 300 going through a central insert 312 and out through cylindrical passages 327 in a base portion 314 of the central insert 312. The top portion of the central insert 312 has an external threaded section which mates with the internal threaded portion 322. Located at the bottom edge of the body 306 and fitted thereto is a seal 308 which is located completely around the outer circumference of the lower edge of body 306 and can also extend internally inside the lower part of the body 306. The outlets 327 can be angled particularly at 45° such that the liquid cryogen enters at a top aperture 328 and exits at a bottom aperture 326 of each outlet 327. The reason for the angled outlets or is so that the liquid cryogen is sprayed at that angle in order to sufficiently cover most of the bud and all the surrounds from between 5 and 25 mm diameter to prevent further horn growth. The outlets or a single outlet can be straight from the capped section especially when horn buds are small.

The seal 308 can be made from silicon, Teflon or rubber however the preferred form is rubber. A vent hole 310 exists in the side of body 306 in order to release the build-up of cryogenic gas, that is, gaseous air or gaseous nitrogen, after the liquid cryogen is vaporized with subsequent heat (cold) transfer. The apparatus 300 connected to the elbow 316 would in turn be connected to a solenoid valve and switch which can be preferably hand held by the user. The outlets 327 can vary in number from one to four and be up to 2 mm in diameter. A preferred value of diameter is 1 to 2.5 mm depending upon the number of outlets.

Shown in FIG. 3E is a further embodiment of another apparatus or application means 330 that is used to apply cryogenic fluid to a site around an existing horn. The horn would protrude through the central open area 331 which extends from a top rim 341 to a bottom rim which is not shown and is covered by a circumferential seal 334. The apparatus 330 includes a vessel 333 having a body 332 which is essentially a hollow cylindrical body open at both ends. Attached, through portions 350 around the inside of the body 332 is a circular ring or tube 346 which is fully enclosed apart from a series of apertures 348 which protrude downwardly into the interior space 331 at a preferable angle of about 45°. The aperture angle can vary from about 10 degrees to 65 degrees with respect to the circular axis of ring 346, according to the apparatus and horn lengths. The angle can be more vertical for longer horns. The Ring 346 is connected to a pair of members 338 and 340 which in turn are connected at the other ends to a connector block or manifold 342. On an opposite side of the manifold 342 there is a connection to an inlet 344 that is threaded which allows cryogenic fluid to flow inwardly to the dispensing arrangement 336 which includes the manifold 342 and the members 338, 340 as well as the ring 346. The threaded inlet 344 connects through a suitable connector, such as a snap-fit connector, to a solenoid valve and the input fluid lines that deliver the liquid nitrogen or air to the apparatus 330.

The seal 334, apart from extending along the outside surface of the body 332 also can extend inwardly against the inside surface of the body 332 in the lower half thereof. It is preferably made from Teflon, silicon or rubber. A handle (not shown) can be attached to the outside of the body 332 with diametrically opposed connection points and this assists in the user pressing the apparatus 330 firmly down on the target site. The other hand of the user can be used to operate a switch to operate a solenoid valve to allow cryogenic fluid into the dispensing device 336. Separation of the ring 346 is needed from the inside of the body 332 (via the connection points 350) in order to limit the amount of heat added to the system, especially when the ring 346 is made from steel. If the ring is a lower heat conductor, such as teflon tubing, then the separation is not as important.

In order to account for varying sizes of the target site, a smaller site can be targeted by inserting a further small apparatus or vessel 350 which has a body 352 and a seal 356 extending around the bottom portion thereof both on the inside and the outside of the body 352. The top rim 354 is simply inserted inside (from below) to interior space 331 of the body 332 of apparatus 330. The rim 354 will be placed in a position below the ring 346 so that liquid cryogen can be directed into the interior space within the body 352 of the apparatus 350. The seal 356 meshes with the seal 334 in order to provide an adequate seal against the escape of the cryogenic fluid. The holes 348 can be angled at about approximately 45 degrees (or between 10 degrees and 65 degrees), downwardly and inwardly with respect to the circular axis of ring 346, in order to target the horn towards its base and flow on to the surrounding soft skin tissues that are located at the horn base. This specifically targets the multiple layers of cells that form the horn base and can extend to a depth of about 10 mm to cryogenically target the growth plate of the ossification centre and can include a width of skin and tissue from a region of between 5 and 25 mm around the base of the horn. The connection of the apparatus 350 to the apparatus 330 can be done by any suitable connection means such as a quick release bayonet fitting and any particular size of apparatus 350 can be used.

As an alternative to using liquid nitrogen as the cryogenic fluid, liquid air can be used that has a boiling point of −170° C. This is slightly higher than the boiling point of liquid nitrogen. The liquid air as a cryogen is cheaper to produce as there is no requirement to filter out nitrogen from air and also where these devices are used on farms often they are located too far away from outlets that dispense liquid nitrogen.

Thus the liquid air or nitrogen is directed at the horn base and the periphery around there. Liquid can run to the outer periphery of the horn base saturating that surrounding area with the cryogenic liquid. The total saturation provides a thorough deep freeze in the targeted site. The cryogenic liquid pressure is preferably a very low pressure in order to keep the fluid as cold as possible. It is generally at around 2 PSI but can be in the range of 1 PSI to 20 PSI. The working pressure can be in the range of 1 to 10 PSI. The lower the pressure then the cooler the cryogenic liquid will be. In hot climates, 10 PSI to 25 PSI may be used to chill and prime the required lines faster. Thereafter the system can operate at between 1 and 10 PSI.

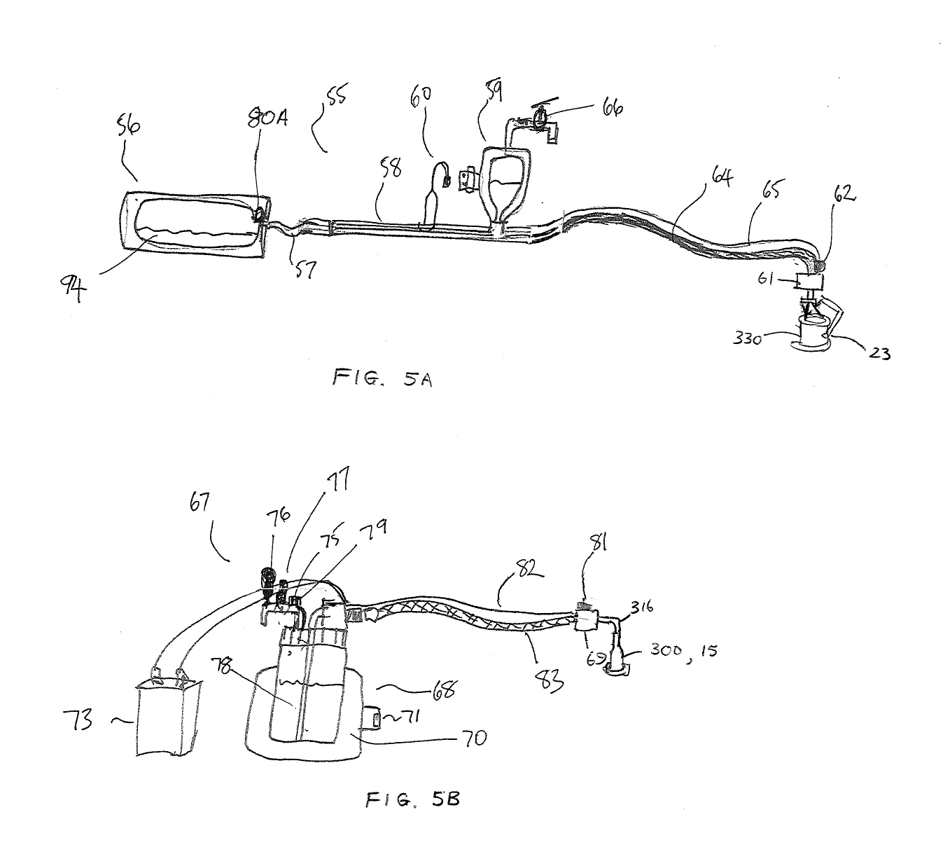

Referring to FIGS. 4A and 4B, there are two embodiments of the apparatus to apply the cryogenic fluid to each of the targeted areas. In FIG. 4A, there is apparatus 15 that applies the liquid nitrogen or liquid air to disperse around and contact the whole horn bud and peripheral skin base area. This would be as in the case of a young calf or lamb or goat. The apparatus 15 is placed squarely over the horn bud 31 and surrounding epidermal layer 32. The outer wall 33 of the open end 19 with flexible seal attached (not shown) provides a barrier to contain the cryogenic liquid and to reduce the risk of the liquid nitrogen/air leaking out from within the defined area. The liquid nitrogen and liquid air 34 is illustrated being dispersed from outlet 21 of internal tube 20. This embodiment of the apparatus 15 is designed to saturate both the horn bud and the surrounding epidermis layer and is utilised for disbudding younger ruminants.

Referring to FIG. 4B, there is an example of an embodiment of an apparatus 23 designed to apply the cryogenic fluid to the surrounding horn epidermal layer and only the outer periphery of the horn bud. This apparatus 23 would be used on young to more mature calves or ruminants and in particular to Bos Indicus type cattle, where the horn bud develops more in an expansive peripheral way with little outward growth as seen in young calves. The horn bud is domed centrally and the overall appearance is oval in shape rather than circular. The centrally located dome 35 aspect of the horn bud acts to deflect the applied cryogenic liquid to the periphery of the horn bud and epidermal skin. This results in the central dome or central section 35 of the horn bud having minimal contact with the cryogenic liquid. The end result being a residual horn bud of variable size and shape, which has been inhibited to grow into a typical horn.

In FIG. 4B, the centrally raised horn bud 35 and peripheral epidermal layer 36 are contained within the elliptical base and conical walled 24 device 23. The cryogenic liquid is transported within the interconnecting tubes, as described previously, and circulates and is dispensed from within the oval-shaped tube 27, which has multiple outlets 30 or 37 mostly on its contact aspect. The cryogenic liquid or in this particular case liquid nitrogen or air is then dispersed within the internally defined area, bordered by the outer rim 38 of the device.

FIGS. 5A and 5B are two examples each showing apparatus to store, deliver, dispense and disperse the liquid cryogenic fluid to the animal's horn bud periphery and/or horn base and its immediate skin circumference.

Referring to FIG. 5A, there is a block diagram of one such example showing apparatus 55 to deliver and apply the liquid cryogenic fluid 94, such as liquid nitrogen or liquid air, to the horn base or horn bud and surrounding immediate skin. Such apparatus 55 can have a variable-sized holding vessel 56 for the liquid nitrogen or liquid air. The vessel 56 is vacuum insulated to hold the cryogenic fluid 94 with temperatures of minus 196 degrees Celsius for nitrogen and minus 170 degrees Celsius for liquid air. As it is anticipated there will be large numbers of animals to perform the process of disbudding or inhibiting horn bud or horn growth, so the holding vessel 56 is required to hold and store up to and more than 450 to 500 kg of the cryogenic fluid. The holding vessel 56 in this particular embodiment is a horizontally placed vessel and is designed for rougher road conditions and can be fitted out in trailers. Smaller upright holding vessels, varying in capacity from 50 to 280 kg of liquid nitrogen or liquid air, can also be used. The vessel 56 has a gate valve 80A, which is connected to a flexible insulated vacuum hose 57 and/or a straight insulated vacuum tube 58. This line can have multiple hoses/tubes interconnected via insulated vacuum connections and the number of such hoses/tubes will depend upon the distance required to transport the cryogenic fluid. Close to the end process of apparatus 55, there is a phase separator vessel 59 that separates out the gaseous component form the liquid component, so the liquid component is always accessible. On the line preceding the phase separator 59 is a thermal relief valve 60. The thermal relief valve 60 is a safety measure designed to release gas built up at very high pressures. The main storage vessel 56 has a variable relief valve 80A that can be set at a range of pressures. In this embodiment for this process, the pressure is set at range of 15 psi to 25 psi. The variable relief valve 80A enables the whole apparatus 55 including the phase separator 59 to operate at the same pre-set pressure. The preferred pressure range is between zero and 40 psi. When not in use, the pressure relief valve 80A can be set at zero. This conserves the release and loss of the cryogenic fluid from pressure build. The phase separator 59 is set up in a vertical position as shown to provide a column of liquid cryogenic fluid, to enable the cryogenic process to have the liquid component effectively immediately on demand. This is important so as to more readily quantify the amount of liquid delivered within a time frame. Thus the time period and depth of freeze improves efficiency in processing time. A release gate valve 66 releases the separated gas component and is situated above the phase separator 59. The solenoid valve 61 is positioned, at the distal end of line or tube 64 adjacent the apparatus 330 or 23 for applying the liquid nitrogen, to draw the liquid component of the liquid nitrogen for example mostly immediately upon activation from the system. The solenoid valve 61 is power-operated and the apparatus depicted is for 24V. The solenoid activation switch can be a hand toggle or push button switch 62. The hand switch 62 can be positioned on or near the horn bud application apparatus 23 or 330. The apparatus 23 or 330 is the end point delivery of the system and applies the cryogenic fluid after being supplied from phase separator 59 through flexible vacuum insulated line 64. The electrical lead 65 for the solenoid valve 61 and hand switch 62 can be integrated with the flexible line 64 transporting the cryogenic fluid. The solenoid valve 61 is located at the distal end of the tube 64 so that there is virtually no loss in temperature of the cryogenic fluid at the point of application.

FIG. 5B is a block view of apparatus 67 to store and dispense smaller, more portable volumes of the cryogenic fluid to the end point of the apparatus 67. The storage vessel 68, which has two steel walls 70 of which the majority of the proportion of these walls 70 are vacuum insulated, with vacuum valve (or cryogenic release valve) 71 and can hold from 0.5 to 25 kg of a cryogenic fluid. Optimal size in capacity of the vessel 68 is 8 to 25 kgs, with a preferred size being 12 kg of the cryogenic fluid. This apparatus 67 has a smaller solenoid valve 69 and switch 81 with a preferred narrower approximate ¼ inch inlet and outlet lines and is located, with a corresponding switch, at the distal end of flexible line 83 of the apparatus 67 adjacent the apparatus 15 or 300 that applies the cryogenic fluid. It is powered by 12 or 24 DC battery 73 via electrical lead or cable 82.

The walls 70 of holding vessel 68 are mostly vacuum-sealed. The lid and upper side aspects of the vessel 68, may not be vacuum-insulated. This non-vacuum area allows heat to penetrate the vessel 68 and promote gassing of the liquid nitrogen/air which, in turn, is designed to create pressure which can be regulated by variable relief valve 77 situated on the one small line 75 connected to the interior of the vessel 68. The pressure created then drives the outflow of the cryogenic liquid from withdrawal tube 78 to the cryogenic solenoid valve 69 and to the targeted application site. This adjustable relief valve 77 enables the system to have regulated pressure control. The pressure is designed to have a range from zero psi to 50 psi maximum and can be set accordingly. The preferred system operating pressure is of the range of 2 psi to 35 psi. On the same line there is also a bursting disc 79 and a manually operated ball valve 76. The bursting disc 79 is designed to release gas pressure at a preset high pressure and this is determined by the engineering capacity and pressure characteristics of the apparatus 67. It is a safety design, in case of excessive pressure build up, resulting in the possibility of the vessel 68 exploding. The cryogenic ball valve 76 function is to reduce the gas build up pressure or vent off the gas pressure when the lid needs to be opened for refilling or say when the device solenoid valve 69 is inoperable, as might be the case when the battery 73 has insufficient power to run the valve 69.

The flexible line 83 generally is vacuum insulated so as not to add too much heat into the system. The distance is determined by several factors including the operator requirements, the set up or animal restraint system, the process used on the animal having the process applied and the actual access length to the equipment. The range can be from 0.5 to 4 m.

In another aspect of the apparatus 67, the cryogenic release valve 71 can be positioned and connected to the holding vessel on the bottom or side wall of a smaller holding vessel like vessel 68 and positioned in a vertical manner (not shown). This will ensure greater reliability of withdrawal of the liquid cryogenic fluid with close proximity to the apparatus 300 or 23.

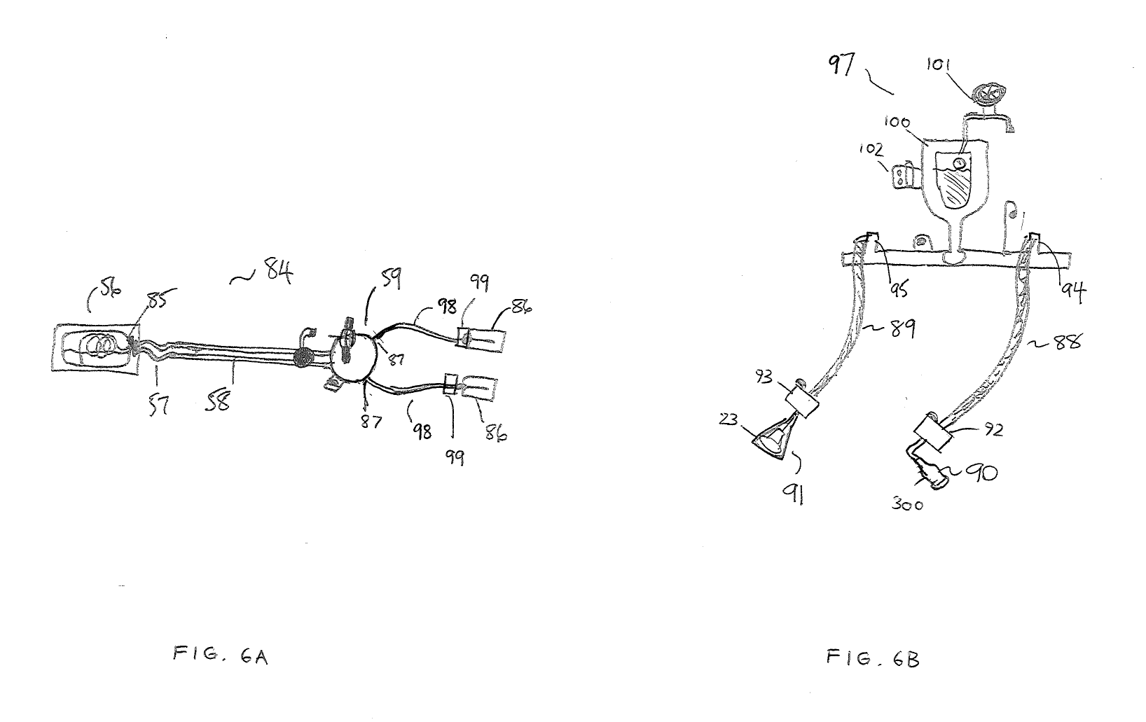

FIG. 6A shows an overhead view of apparatus 84 and delivery system similar to that of FIG. 5A. There is a large holding vessel 56 which is vacuum insulated, a gate valve 85 and flexible vacuum-insulated outlet pipe 57 and rigid vacuum-insulated pipe lines 58 connected to a phase separator 59. There are two hoses 98 which are connected to application means 86 for applying the cryogenic fluid associated with the disbudding of small horn buds. In this embodiment there are two lines and application means so both horns can be processed at the same time. Alternatively, a slightly varying size application means can be utilised on the one line so there is a choice for the operator to choose the most appropriate application means size according to the horn bud circumference presented. This gives important system flexibility to allow for ranges in animal age, size and horn or horn bud size. There are two outlets 87 from the phase separator 59 and in this embodiment, are at 90 degrees to one another. The outlets 87 are insulated bayonet attachments connected to vacuum insulated flexible lines 98 and then to solenoid valves 99 that can be connected directly to the horn bud application apparatuses 86. Solenoid control is via finger button in the vicinity of the horn bud application apparatuses 86.

Referring to FIG. 6B, there is another embodiment of the apparatus 97 and delivery system with two end point lines 88 and 89. The two flexible lines transport the cryogenic fluid to the application apparatuses 90 and 91. The lines 88 and 89 are connected to the two independent solenoid valves 92 and 93, which in turn are connected to the phase separator 100, so as to deliver mostly the liquid component of the cryogenic fluid. There are two small risers or insulated bayonets 94 and 95 which are connected via vacuum insulated lines to connect to the inlet of the solenoid valves 92 and 93 respectively. Generally these risers or bayonets 94, 95 are steel pipes and are vacuum insulated so as to minimise heat transfer. As the solenoid valves 92, 93 cannot be fully vacuum insulated, they can add heat to the system and produce gas internally within the line. The gas produced in this area escapes back to the phase separator 100 and is released by the opened relief valve 101. Upon activation, the solenoid valves 92, 93 will tend to instantaneously release mostly cryogenic liquid. The distance between the two-solenoid valves 92, 93 can vary with each system. For processing horn and horn buds, the preferred distance is approximately 40 cm and can range from 30 cm to 2 m, with the line length being 3 to 6 m. The differing apparatus 91 and 300 are valuable to be available at the time of mustering large numbers of cattle. This embodiment would be valuable for example in the northern pastoral parts of Australia, where the stock are often mustered once a year and where the cows breed throughout the year. So there is a year's range of horn bud and horn sizes in the younger stock. Some animals are missed the first year and may be mustered the following year, in which case the application used through this invention on these cattle would be effective in inhibiting further growth and diminishing the integrity and strength of the yearling animals' horns. This saves the need to cut the horn off as is sometimes practiced.

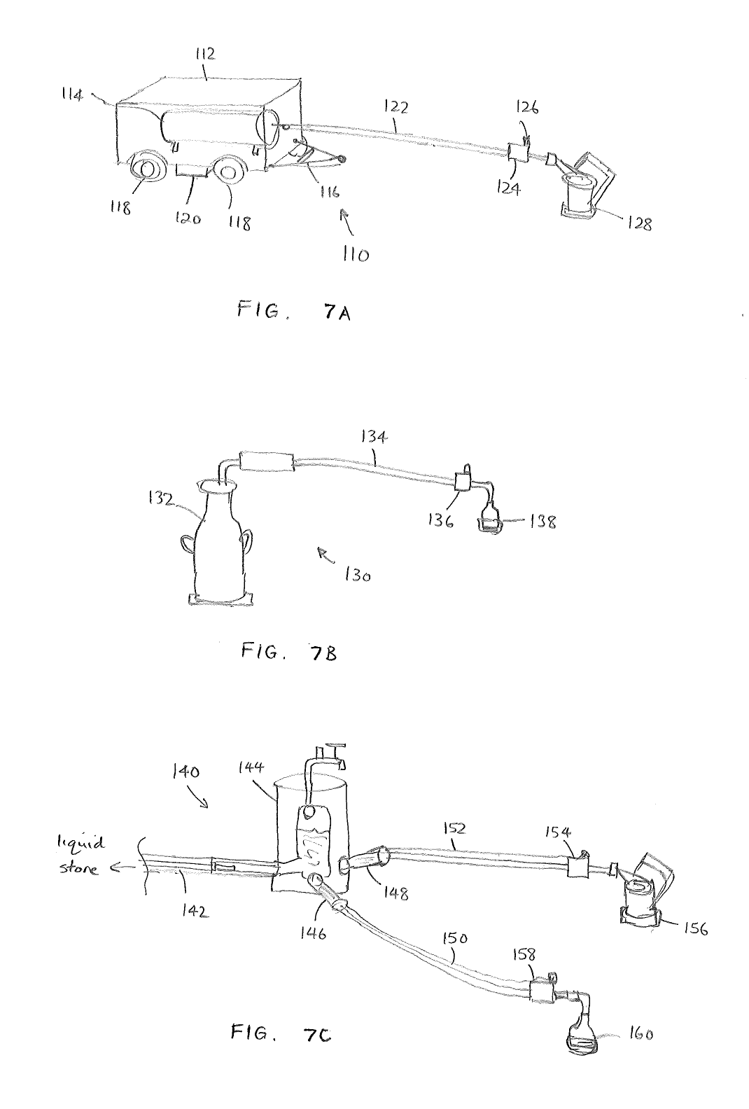

Referring to FIG. 7A there is shown a system 110 for delivering cryogenic fluid to an apparatus 128 for applying the fluid to a target site around a horn bud or horn of a livestock animal. It includes a pressure holding vessel 114 for holding the liquid cryogen and this is mounted to a trailer 112 or other portable vehicle. A hitch and frame assembly 116 is at the front of the trailer 112 which has standard wheels 118 and a lift point 120. A vacuum line 122 extends from the vessel 114 to the apparatus 128 which is hand-operated by a user and controlled using a solenoid valve 124 with switch 126 for controlling the solenoid valve.

Shown in FIG. 7B is a smaller system 130 for delivering the cryogenic fluid. It is delivered from a portable holding vessel 132 that holds liquid nitrogen or liquid air and this is distributed to the end apparatus 138 that has a solenoid valve 136 and switch for control by the user. It is delivered over the vacuum line 134.

Shown in FIG. 7C is a further system 140 for delivering cryogenic fluid to multiple outputs from the one delivery station. The cryogenic fluid is distributed along lines 142 from a holding storage vessel (not shown) to a phase separator 144. By using a phase separator it delivers the liquid cryogen by eliminating the gas component, which makes the system more efficient and improving the process control. The phase separator operates in a standard way by splitting the gas phase from the liquid phase of the cryogen and adjusting the pressure in the vessel through a float valve which releases a build up of gas at the appropriate time. Any number of outlets or connectors 146 and 148, which are bayonet vacuum insulated connectors, can be used to distribute the cryogenic fluid along different lines. For example from outlet 146 distributed along line 150 is liquid cryogen delivered to apparatus 160 for applying to a livestock animal. A corresponding solenoid valve 158 is located near the apparatus 160 so that it can be operated through the user's hands. Similarly from outlet 148 there is a supply line 152 with appropriate electrical leads to the solenoid valve 154 or the electrical lines can be disassociated from the cryogenic supply lines. The liquid cryogen is delivered to the apparatus 156, which is for example the embodiment 330 shown in FIG. 3E which is hand held by the user. The apparatuses 160 or 156 are inter-changeable through appropriate connection means to cater for the different applications of the liquid cryogen.

1. A method of inhibiting the growth of a horn or a horn bud in a livestock animal including the steps of:
applying a cryogenic fluid around the base of the horn or on the horn bud using an apparatus;
such that the cryogenic fluid contacts the base of the horn and epidermal skin layers that surrounds the horn or horn bud and induces a freeze in said layers to inhibit or prevent any further growth of the horn or horn bud.

2. A method according to claim 1 wherein the application of the cryogenic fluid to said horn base and skin layers results in a freezing and thawing of cells and tissues that make up said layers and thereby reducing or eliminating the vascular, cellular and neural integrity of the layers.

3. A method according to claim 1 or claim 2 wherein the cryogenic fluid contacting the layers is either in liquid form, gaseous form or a combination of a liquid and gaseous form.

4. A method according to any one of the preceding claims wherein the surrounding horn base and skin layers is subjected to a cryogenic fluid temperature of between minus 20 degrees Celsius and minus 80 degrees Celsius, preferably minus 50 degrees Celsius.

5. A method according to any one of the preceding claims further including applying the cryogenic fluid to the whole of the horn bud in a young livestock animal.

6. A method according to any one of the preceding claims wherein said horn base and surrounding epidermal skin layers after application of the cryogenic fluid, undergoes a healing process where there is a closed wound and where cellular necrosis and scar formation results.

7. A method according to any one of the preceding claims wherein the targeted skin layers surrounding the horn or horn bud is up to 25 mm wide.

8. A method according to any one of the preceding claims wherein said cryogenic fluid is liquid nitrogen or liquid air.

9. A method according to any one of the preceding claims further including determining the amount of cryogenic fluid to use based on the size of the horn or horn bud and the age, breed and size of the livestock animal.

10. Apparatus for inhibiting the growth of a horn or a horn bud in a livestock animal including:
a vessel having a body through which a cryogenic fluid is applied around the base of the horn or on the horn bud;
such that the cryogenic fluid contacts the horn base and epidermal skin layers that surrounds the horn or horn bud and induce a freeze in said layers to inhibit or prevent any further growth of the horn or horn bud.

11. Apparatus according to claim 10 further including one or more apertures or passages through which the cryogenic fluid passes and is directed onto the horn base and the skin surrounding said horn or horn bud.

12. Apparatus according to claim 10 or claim 11 wherein said body is open at an end which is placed over the horn or the horn bud.

13. Apparatus according to claim 12 further including seal means formed around said end to minimize or prevent the escape of the cryogenic fluid and to assist in directing the cryogenic fluid to said skin layers.

14. Apparatus according to claim 13 wherein an opposite end to said end has connected thereto the supply of cryogenic fluid to the apparatus.

15. Apparatus according to claim 14 further including a vent hole to release any excess gas released from vaporization of the cryogenic fluid from within the body.

16. Apparatus according to claim 14 or claim 15 further including an interior member housing one or more outlets through which the cryogenic fluid flows, said one or more outlets angled so as to deliver the cryogenic fluid directly to the horn bud and the skin layers around and on said horn bud.

17. Apparatus according to claim 13 wherein an opposite end to said end is also open to enable the horn of the animal to protrude.

18. Apparatus according to claim 17 further including a ring to fit within the confines of the body, said ring having one or more apertures through which flows the cryogenic material.

19. Apparatus according to claim 18 wherein said one or more apertures are directed downwardly at between 10 degrees and 65 degrees, preferably at about 45 degrees, with respect to the circular axis of the ring in order to target the skin layers with the cryogenic fluid.

20. Apparatus according to claim 19 further including a manifold, one side of which is connected to a supply of the cryogenic fluid and an opposite side connected to one or more members that are also connected to said ring to deliver the cryogenic fluid to the ring.

21. Apparatus according to claim 20 wherein the ring is spaced apart from an interior of the body.

22. Apparatus according to any one of claims 17 to 21 further including handle means to assist a user in applying the apparatus to the animal with sufficient force.

23. Apparatus according to claim 22 wherein a further apparatus having a body smaller in diameter to said apparatus and also having a seal means is able to be fitted within said apparatus and below the ring in order to accommodate for a different sized horn.

24. Apparatus according to any one of claims 10 to 23 wherein said cryogenic fluid is applied for between 1 second and 10 seconds.
